SEQUENCE LISTING (2 sequences)

SEQ ID NO: 1 - DNA, 180 bp, Mus musculus
atggatgtgttcatgaaaggactttcaaaggccaaggagggagttgtggctgctgctgagaaaaccaagcagggtgtggcagaggcagctggaaagacaaaagagggagtcctctatgtaggttccaaaactaaggaaggagtggttcatggagtgacaacagtggctgagaagaccaaa

SEQ ID NO: 2 - protein, 60 aa, Mus musculus
MDVFMKGLSKAKEGVVAAAEKTKQGVAEAAGKTKEGVLYVGSKTKEGVVHGVTTVAEKTK

(19) United States
(12) Patent Application Publication
(10) Pub. No.: US 2021/0002712 A1
(43) Pub. Date: Jan. 7, 2021

(54) PHOSPHATIDIC ACID SENSOR

(71) Applicant: NATIONAL UNIVERSITY CORPORATION CHIBA UNIVERSITY, Chiba (JP)

(72) Inventor: Fumio SAKANE, Chiba (JP)

(21) Appl. No.: 16/920,269

(22) Filed: Jul. 2, 2020

(30) Foreign Application Priority Data
Jul. 5, 2019 (JP) 2019-126050
Feb. 7, 2020 (JP) 2020-019443

(51) Int. Cl. C12Q 1/6827; C12Q 1/6837

(52) U.S. Cl. CPC C12Q 1/6827; C12Q 2600/156; C12Q 1/6837

(57) ABSTRACT

Disclosed is a phosphatidic acid visualizing sensor configured to detect phosphatidic acid produced in a cell with high accuracy. The phosphatidic acid sensor an N-terminal region of α-synuclein. Furthermore, the phosphatidic acid sensor includes a peptide having any one of following amino acid sequences (1) to (3):
(1) an amino acid sequence of SEQ ID No. 2;
(2) an amino acid sequence having 90% or more homology to the amino acid sequence of SEQ ID No. 2; and
(3) an amino acid sequence in which 6 or less amino acids are deleted, substituted, and/or added to the amino acid sequence of SEQ ID No. 2.

### TECHNICAL FIELD

The present invention relates to a phosphatidic acid sensor.

### BACKGROUND ART

In the background art, phosphatidic acid (PA) is the simplest phospholipid existing in biological membranes. Although PA constitutes only a minor fraction of total cell lipids, it is involved in the regulation of a wide variety of biological phenomena, including mitogenesis, migration and differentiation. Many reports have demonstrated that PA controls a number of signaling proteins, such as phosphatidylinositol (PI)-4-phosphate 5-kinase (PIP5K), mammalian target of rapamycin (mTOR), atypical protein kinase C (αpkc), and p21 activated protein kinase 1. Moreover, the amount of PA is spaciotemporally and strictly regulated. For example, PA was greatly increased during the differentiation of neuroblastoma and pheochromocytoma cells and enriched in synaptic ribbon. In addition, PA level changes correlate with protein transit and phagocytosis.

PA is generated from multiple pathways. PA, as an intracellular signaling lipid, is primarily generated by the phosphorylation of diacylglycerol (DG) by DG kinase (DGK) and by the hydrolysis of phosphatidylcholine (PC) by phospholipase D (PLD). Ten DGK isozymes (DGKα, β, δ, γ, η, κ, ε, ζ, τ, and θ) have been identified, and they are involved in the pathogenesis of a wide variety of diseases, such as cancer, epilepsy, obsessive-compulsive disorder, bipolar disorder, autoimmunity, cardiac hypertrophy, hypertension and type II diabetes.

PLD also consists of 2 isozymes (PLD1 and 2) and is related to various diseases, such as cancer and neurodegenerative disorders, including Parkinson's and Alzheimer's diseases. In addition, PA, which serves as a critical intermediate of cell membrane phospholipid de novo synthesis, is produced by lysoPA (LPA) acyltransferase (LPAAT). LPAAT is also a therapeutic target for ovarian and endometrial cancers and acute leukemia.

A phosphatidic acid sensor is referred to Non-Patent Citation 1.

### CITATION LIST

### Non-Patent Literature

Non-Patent Citation 1: Comparative Characterization of Phosphatidic Acid Sensors and Their Localization during Frustrated Phagocytosis” THE JOURNAL OF BIOLOGICAL CHEMISTRY VOL. 292, NO. 10, pp. 4266-4279, Mar. 10, 2017

### SUMMARY OF INVENTION

### Technical Problem

A phosphatidic acid-binding protein/domain described in Non-patent literature 1, which intrinsically localizes to the endomembrane, localizes to the membrane regions (cytoplasm, Golgi apparatus, etc.) in a cell even when phosphatidic acid is not produced. That is, there is a problem in that a high level of background activity makes it difficult to precisely detect intracellular phosphatidic acid produced in the endomembrane when the cell is stimulated or a phosphatidic acid-producing enzyme is expressed.

Thus, an object of the present invention is to provide a phosphatidic acid visualizing sensor capable of detecting phosphatidic acid produced in a cell with high accuracy.

### Solution to Problem

In order to solve the foregoing problems, a phosphatidic acid sensor comprising an N-terminal region of α-synuclein.

Furthermore, the phosphatidic acid sensor comprising a peptide having an amino acid sequence encoded by any one of following base sequences (1) to (3):
(1) a base sequence of SEQ ID No. 1;
(2) a base sequence having 90% or more homology to the base sequence of SEQ ID No. 1; and
(3) a base sequence in which 18 or less bases are deleted, substituted, and/or added to the base sequence of SEQ ID No. 1.

Furthermore, the phosphatidic acid sensor comprising a peptide having any one of following amino acid sequences (1) to (3):
(1) an amino acid sequence of SEQ ID No. 2;
(2) an amino acid sequence having 90% or more homology to the amino acid sequence of SEQ ID No. 2; and
(3) an amino acid sequence in which 6 or less amino acids are deleted, substituted, and/or added to the amino acid sequence of SEQ ID No. 2.

### Advantageous Effect of the Invention

According to the aforesaid characteristic, a phosphatidic acid sensor is capable of detecting phosphatidic acid produced in a cell.

### BRIEF DESCRIPTION OF DRAWINGS

FIG. 1 is a schematic architecture of α-Syn and its deletion mutants.

FIG. 2 shows the purified SUMO-tagged-α-Syn and its deletion mutant protein (2.5 μM) were incubated with 18:1/18:1-PA or 18:1/18:1-PS liposomes (PA or PA: 150 μM) and then separated by ultracentrifugation.

FIG. 3 shows the purified SUMO-tagged-α-Syn-N protein (2.5 μM) was incubated with control (PC and Chol alone), 16:0/16:0-PA, 16:0/18:1-PA, 18:1/18:1-PA, or 18:0/18:0-PA liposomes (PA: 150 μM) and then separated by ultracentrifugation.

FIG. 4 shows the amounts of protein in the supernatant (S) and precipitant (P) were quantified by densitometry using ImageJ software.

FIG. 5 shows the purified SUMO-tagged-α-Syn protein (0.1 μM) was incubated with 18:1/18:1-PA liposomes of the indicated concentrations (0-20 μM) and then separated by ultracentrifugation. SDS-PAGE (15%) was performed and separated proteins were detected with silver staining.

FIG. 6 shows the amounts of protein in the precipitant were quantified by densitometry using ImageJ software. Binding activity was calculated as the percentage of the precipitate band intensity compared to the total band intensities (Input).

FIG. 7 shows the purified SUMO-tagged-α-Syn protein (0.1 μM) was incubated with 18:1/18:1-PA, 18:1/18:1-PS, 18:1/18:1-PI(4,5)P₂, 18:1/18:1-PE and d18:1/18:1-C1P liposomes (20 μM) and then separated by ultracentrifugation. SDS-PAGE (15%) analysis was performed and separated proteins were detected with silver staining.

FIG. 8 shows the amounts of protein in the precipitant were quantified by densitometry using ImageJ software. The PA binding activity of α-Syn-N was set to 100%.

FIG. 9 shows equimolar amounts (100 pmol) of various lipids were spotted onto nitrocellulose membranes as indicated.

FIG. 10 shows the blots were scanned and quantified using ImageJ software. The PA binding activity (spot intensity) of α-Syn-N was set to 100%.

FIG. 11 shows equimolar amounts (300 pmol) of 18:1/18:1-PA, 18:1-LPA, and d18:1/18:1-C1P were spotted onto nitrocellulose membranes as indicated.

FIG. 12 shows the blots were scanned and quantified using ImageJ software. The PA binding activity (spot intensity) of α-Syn-N was set to 100%.

FIG. 13 shows EGFP alone, EGFP-DGKβ-WT or EGFP-DGKβ-KD was coexpressed with either the DsRed monomer alone or DsRed monomer-α-Syn-N in COS-7 cells. After 24 h of transfection, the cells were fixed and imaged.

FIG. 14 show the localization of EGFP-DGKβ-WT, EGFP-DGKβ-KD and DsRed monomer-α-Syn-N was quantified using ImageJ software.

FIG. 15 shows EGFP-DGKβ-WT was coexpressed with DsRed monomer-α-Syn-N in COS-7 cells. After 24 h of transfection, the cells were incubated with 1 μM Compound A in growth medium for 1 h, fixed and imaged.

FIG. 16 show the localization of EGFP-DGKβ-WT and DsRed monomer-α-Syn-N was quantified using ImageJ software.

FIG. 17 shows either Myr-AcGFP-DGKζ-WT or Myr-AcGFP-DGKζ-KD was coexpressed with either the DsRed monomer alone or DsRed monomer-α-Syn-N in COS-7 cells as indicated. After 24 h of transfection, the cells were fixed and imaged.

FIG. 18 shows the localization of Myr-AcGFP-DGKζ-WT, Myr-AcGFP-DGKζ-KD and DsRed monomer α-Syn-N was quantified using ImageJ software.

FIG. 19 shows either EGFP alone, EGFP-DGKγ-WT or EGFP-DGKγ-KD was coexpressed with either the DsRed monomer alone or DsRed monomer-α-Syn-N in COS-7 cells as indicated. After 24 h of transfection, the cells were incubated with 1 μM PMA or DMSO alone in growth medium for 30 min, fixed and imaged.

FIG. 20 show the localization of EGFP-DGKγ-WT, EGFP-DGKγ-KD and DsRed monomer α-Syn-N was quantified using ImageJ software.

FIG. 21 shows EGFP-PLD was coexpressed with either the DsRed monomer alone or DsRed monomer α-Syn-N in COS-7 cells as indicated. After 24 h of transfection, the cells were incubated with 750 nM FIPI in growth medium for 4 h and then fixed and imaged.

FIG. 22 show the localization of EGFP-PLD2-WT and DsRed monomer α-Syn-N was quantified using ImageJ software.

FIG. 23 shows either EGFP alone or EGFP-PIP5K1A was coexpressed with either the DsRed monomer alone, DsRed monomer α-Syn-N or DsRed monomer PLCδ1-PHD in COS-7 cells as indicated. After 24 h of transfection, the cells were fixed and imaged.

FIG. 24 show the localization of EGFP-PIP5K1A, DsRed monomer α-Syn-N and DsRed monomer-PLCδ1-PHD was quantified using ImageJ software.

FIG. 25 shows SDS-PAGE (15%) analysis of GST or SUMO-tagged α-Syn and its mutants expressed in E. coli and purified by affinity chromatography.

FIG. 26 shows SDS-PAGE (15%) analysis of GST or SUMO-tagged α-Syn and its mutants expressed in E. coli and purified by affinity chromatography.

FIG. 27 show equimolar amounts (500 pmol) of 18:1/18:1-PA, 18:1/18:1-PC, 18:1/18:1-PE, 18:1/18:1-PG), 18:1/18:1-PS, 18:1/18:1-PI and 18:1/18:1-PI(4,5)P₂ were spotted onto nitrocellulose membranes as indicated and incubated with purified GST-tagged-α-Syn-N(20 nM).

FIG. 28 shows the blots were scanned and quantified using ImageJ software. The PA binding activity (spot intensity) of α-Syn-N was set to 100%.

FIG. 29 shows either EGFP alone or EGFP-DGKβ was coexpressed with the DsRed monomer alone, DsRed monomer-Spo20p-PABD or DsRed monomer-PDE4A1-PABD in COS-7 cells as indicated. After 24 h of transfection, the cells were fixed and imaged.

FIG. 30 show Myr-AcGFP-DGKζ and/or DsRed monomer-PDE4A1-PABD were expressed in COS-7 cells as indicated. After 24 h of transfection, the cells expressing Myr-AcGFP-DGKζ alone were immunostained with an anti-TGN46 antibody and an Alexa Fluor 568-conjugated secondary antibody.

FIG. 31 show Myr-AcGFP-DGKζ and/or DsRed monomer-PDE4A1-PABD were expressed in COS-7 cells as indicated. After 24 h of transfection, the cells expressing Myr-AcGFP-DGKζ alone were immunostained with an anti-TGN46 antibody and an Alexa Fluor 568-conjugated secondary antibody.

FIG. 32 shows either Myr-AcGFP-DGKζ alone or Myr-AcGFP-DGKζ-KD was coexpressed with the DsRed monomer alone or DsRed monomer-Spo20p-PABD in COS-7 cells as indicated. After 24 h of transfection, the cells were fixed and imaged.

FIG. 33 shows either Myr-AcGFP-DGKζ alone or Myr-AcGFP-DGKζ-KD was coexpressed with the DsRed monomer alone or DsRed monomer-Spo20p-PABD in COS-7 cells as indicated. After 24 h of transfection, the cells were fixed and imaged.

FIG. 34 show EGFP-PLD2 and/or DsRed monomer-PDE4A1-PABD were expressed in COS-7 cells as indicated. After 24 h of transfection, the cells expressing Myr-AcGFP-DGKζ alone were immunostained with an anti-TGN46 antibody and an Alexa Fluor 594-conjugated secondary antibody.

FIG. 35 show EGFP-PLD2 and/or DsRed monomer-PDE4A1-PABD were expressed in COS-7 cells as indicated. After 24 h of transfection, the cells expressing Myr-AcGFP-DGKζ alone were immunostained with an anti-TGN46 antibody and an Alexa Fluor 594-conjugated secondary antibody.

### DESCRIPTION OF EMBODIMENTS

One object of the present invention, a phosphatidic acid (visualization) sensor comprises an N-terminal region of α-synuclein.

A phosphatidic acid sensor comprising an N-terminal region of α-synuclein.

Furthermore, the phosphatidic acid sensor comprising a peptide having an amino acid sequence encoded by any one of following base sequences (1) to (3):
(1) a base sequence of SEQ ID No. 1;
(2) a base sequence having 90% or more homology to the base sequence of SEQ ID No. 1; and
(3) a base sequence in which 18 or less bases are deleted, substituted, and/or added to the base sequence of SEQ ID No. 1.

Furthermore, the phosphatidic acid sensor comprising a peptide having any one of following amino acid sequences (1) to (3):
(1) an amino acid sequence of SEQ ID No. 2;
(2) an amino acid sequence having 90% or more homology to the amino acid sequence of SEQ ID No. 2; and
(3) an amino acid sequence in which 6 or less amino acids are deleted, substituted, and/or added to the amino acid sequence of SEQ ID No. 2.

### First Embodiment

### 1. Introduction

Tracking the localization and dynamics of intracellular PA is essential for understanding a wide variety of biological phenomena regulated by PA. Several PA-binding domains (PABDs), such as sporulation-specific protein 20 (Spo20p)-PABD and cAMP phosphodiesterase-4A1 (PDE4A1)-PABD, are often used as PA sensors. However, they exhibit their own subcellular localization to the plasma membrane (Spo20p-PABD) and Golgi apparatus (PDE4A1-PABD) in a cell stimulation-independent manner (a cell stimulation-induced PA generation-independent manner). The cell stimulation-independent localization disturbs their functions as PA sensors and, consequently, makes them difficult to apply. Therefore, a reliable and widely applicable PA sensor that can be used for any cell stimulation and cell type has not been developed to date.

Recently, we found that α-Synuclein (α-Syn) strongly and selectively bound to PA (34). Therefore, we explored the possibility of α-Syn as a promising PA sensor. The N-terminal region of α-Syn (α-Syn-N) strongly bound to PA in vitro. α-Syn-N did not show its own membrane localization. The sequence of the N-terminal region of α-Syn is shown in SEQ ID NO:1. The amino acid sequence encoded by the base sequence shown in SEQ ID NO: 1 is shown in SEQ ID NO: 2. Moreover, α-Syn-N colocalized with PA-generating enzymes, such as DGK and PLD, but not with a PI 4,5-bisphosphate (PI(4,5)P2)-producing enzyme, PIP5K, in an activity-dependent manner, indicating that α-Syn-N selectively binds to intracellular PA. Therefore, α-Syn-N could be utilized as a reliable and widely applicable PA sensor in cells.

### 2. Results

### (a) PA-Binding Activity of α-Syn and its Deletion Mutants

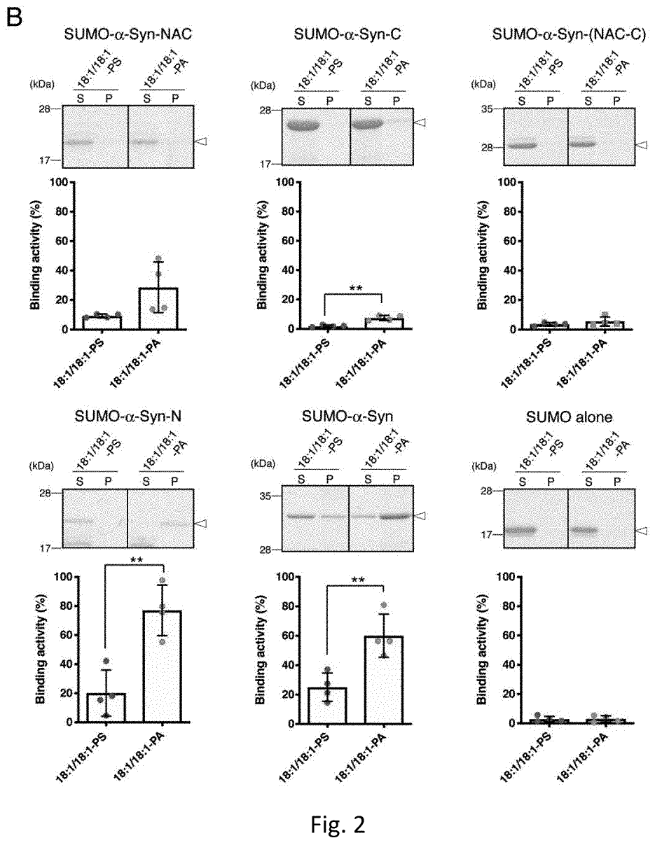

We first performed a liposome cosedimentation assay of 6×His-SUMO-tagged α-Syn and its deletion mutants, the N-terminal region (α-Syn-N), the non-amyloid-β-component (α-Syn-NAC), the C-terminal region (α-Syn-C), and NAC and C (α-Syn-NAC-C), using 18:1/18:1-PA, to which full-length α-Syn strongly bound. As shown in FIG. 2, α-Syn-N most strongly bound to 18:1/18:1-PA, whereas α-Syn-NAC, α-Syn-C, α-Syn-NAC-C and 6×His-SUMO alone failed to show substantial PA-binding activity. Moreover, the PA binding activity of α-Syn-N was comparable with that of full-length α-Syn.

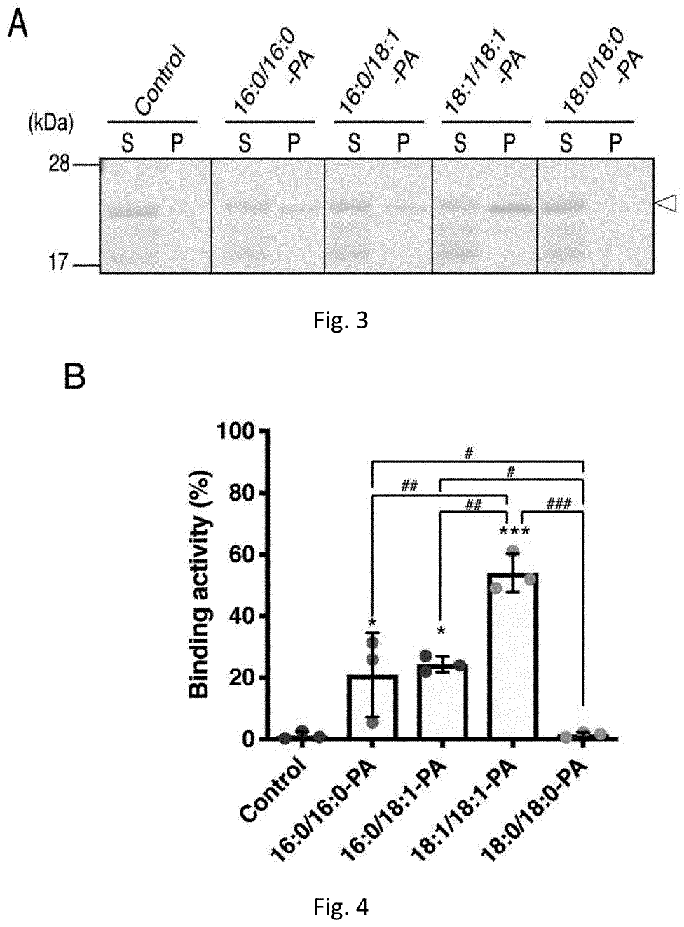

Liposome cosedimentation assays of α-Syn-N using various PA molecular species, 16:0/16:0-, 16:0/18:1-, 18:1/18:1- and 18:0/18:0-PA, were carried out. α-Syn-N most strongly bound to 18:1/18:1-PA and considerably interacted with 16:0/16:0- and 16:0/18:1-PA (FIGS. 3,4), consistent with our previous results using full-length α-Syn.

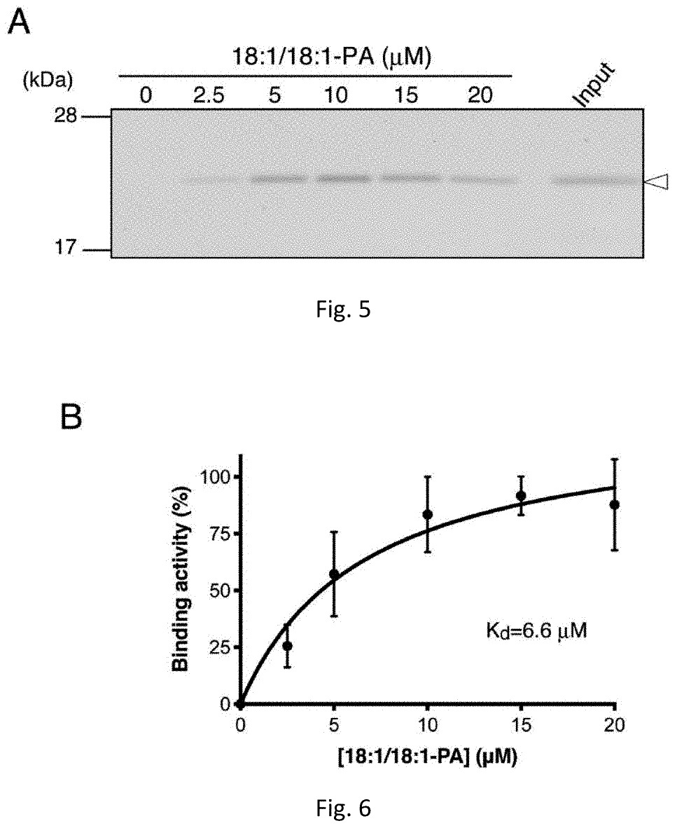

The affinity of α-Syn-N for PA (18:1/18:1-PA) was determined using a liposome cosedimentation assay with various concentrations of 18:1/18:1-PA. A PA concentration-dependent increase in liposome cosedimentation of α-Syn-N was observed and the dissociation constant (K_{d}) was determined to be 6.6 μM. This value is comparable with other PABDs, including Spo20p-PABD (K_{d}=2.2 μM) and PDE4A1-PABD (K_{d}=6.8 μM) (FIGS. 5,6).

We compared the affinities of α-Syn-N for acidic glycerolipids, 18:1/18:1-PA, 18:1/18:1-phosphatidylserine (PS) and 18:1/18:1-PI(4,5)P₂ using a liposome cosedimentation assay. As shown in FIGS. 6,7, α-Syn-N most strongly bound to 18:1/18:1-PA, whereas only weak signals were detected with PI(4,5)P₂ and no signals were observed with PS.

Inclusion of conical lipids such as PA, phosphatidylethanolamine (PE) or ceramide-1-phosphate (C1P), with a relatively small size of their head group, imposes negative membrane curvature. Thus, we next performed a liposome cosedimentation assay using PE and C1P. α-Syn-N did not substantially interact with PE or C1P (FIG. 8). Therefore, it is likely that the PA binding activity of the domain is PA selective but not curvature sensitive.

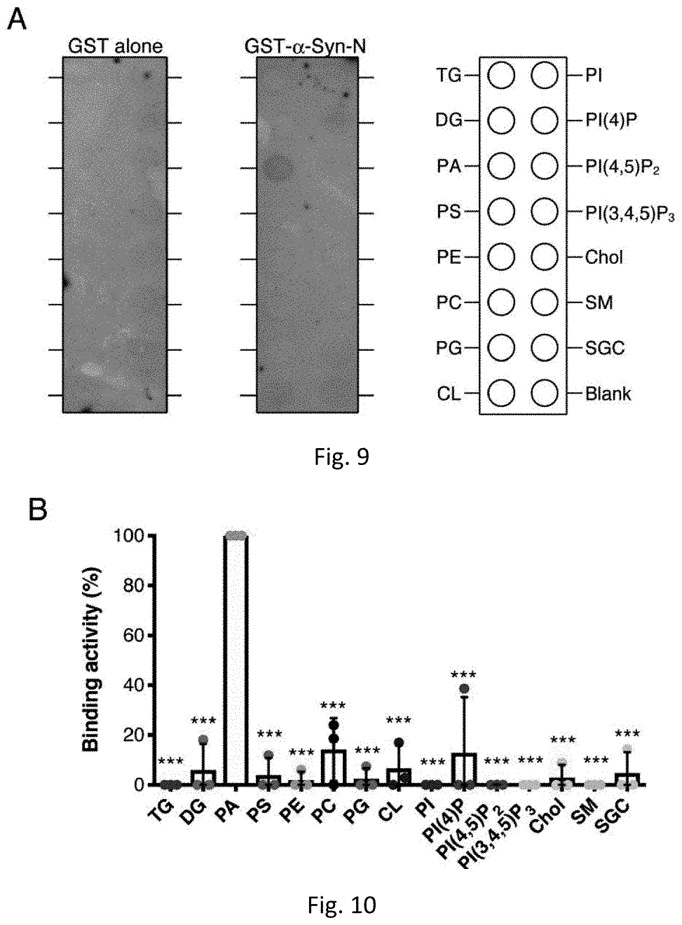

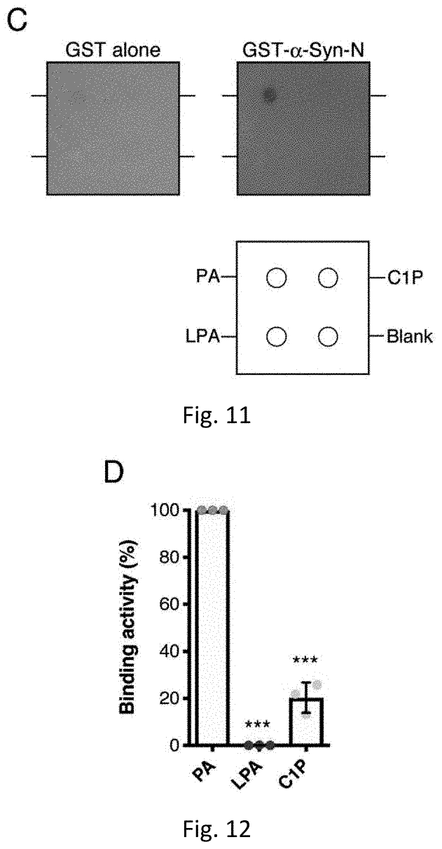

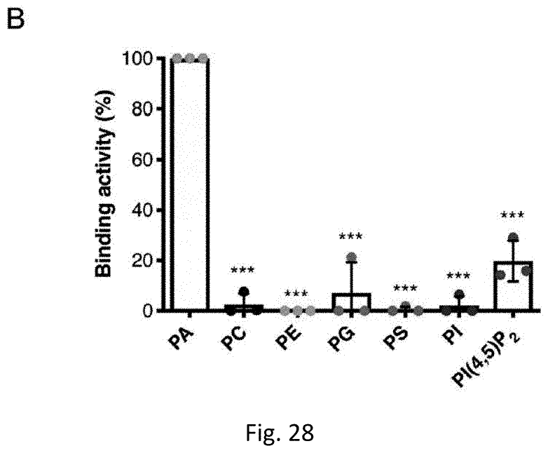

To further analyze the lipid selectivity of α-Syn-N, a lipid overlay assay was performed using a membrane spotted with triacylglycerol (TG), DG, PA, PS, PE, PC, phosphatidylglycerol (PG), cardiolipin (CL), PI, PI(4)P, PI(4,5)P₂, PI(3,4,5)P₃, cholesterol (Chol), sphingomyelin (SM) and sulfo-galactosylceramide (SGC). Among them, PA, PG, PS, PI, PI(4)P, PI(4,5)P₂, PI(3,4,5)P₃ and SGC are acidic lipids. Because 6×His-SUMO tag gave high background staining in lipid overlay assays, we changed to glutathione S-transferase (GST) tag. As shown in FIGS. 9, 10, GST-fused α-Syn-N most strongly bound to PA, whereas only weak or no signals were detected with other lipids. In this experiment, 16:0/16:0-glycerolipids were used. However, essentially the same results were obtained using 18:1/18:1-PA, 18:1/18:1-PC, 18:1/18:1-PE, 18:1/18:1-PG, 18:1/18:1-PS, 18:1/18:1-PI and 18:1/18:1-PI(4,5)P₂ (FIGS. 27, 28). Moreover, PA exhibited significantly stronger binding to α-Syn-N than LPA and C1P, which are acidic phospholipids (FIGS. 11, 12). Taken together, these results indicate that α-Syn-N selectively binds to PA.

### (2) Colocalization of α-Syn-N with DGKβ at the Plasma Membrane in COS-7 Cells

We next determined whether α-Syn-N colocalized with the PA-generating enzymes DGK and PLD. We first verified that DsRed monomer-tagged α-Syn-N showed a broad distribution in the cytoplasm and the nucleus, which was essentially the same as the DsRed monomer alone, indicating that α-Syn-N does not have its own subcellular distribution ability to membranes, such as the plasma membrane and Golgi apparatus.

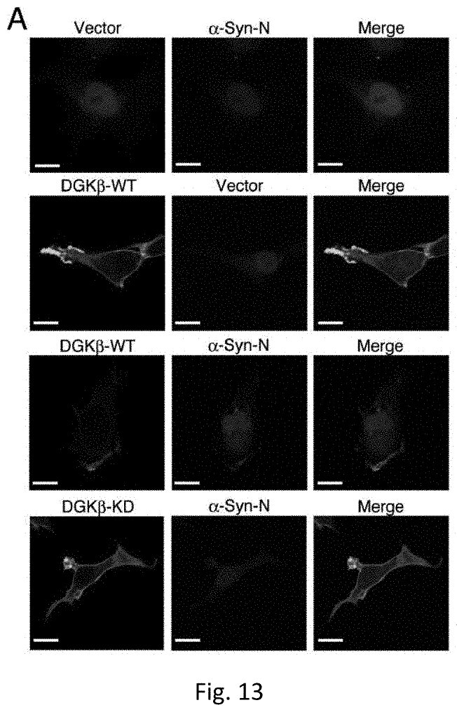

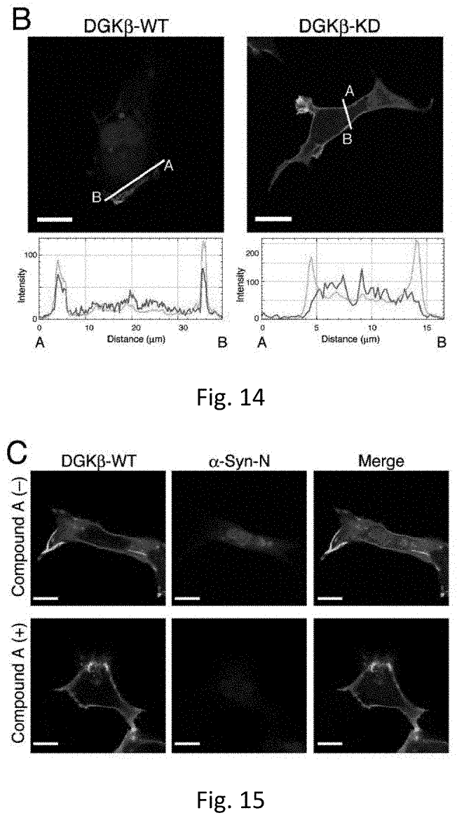

DGKβ is known to localize primarily at the plasma membrane where its substrate, DG, exists. We confirmed that EGFP-tagged DGKβ was obviously distributed at the plasma membrane in COS-7 cells (FIG. 13). Moreover, we found that DsRed-α-Syn-N clearly colocalized with EGFP-DGKβ(FIGS. 13, 14). However, EGFP-DGKβ-KD, a kinase-dead inactive mutant, was not colocalized with DsRed-α-Syn-N. An inhibitor of type I DGK isozymes (DGKα, β and γ), Compound A (IC50 for DGKβ: 0.02 μM), nullified the colocalization of EGFP-DGKβ and DsRed-α-Syn-N, indicating that the colocalization occurred in a DGK activity (PA)-dependent manner and that α-Syn-N does not recognize the DGKβ protein itself. These results strongly suggest that α-Syn-N can detect PA produced by DGKβ in the plasma membrane.

We determined whether the localization of Spo20p-PABD and PDE4A1-PABD overlapped with that of DGKβ. DsRed-Spo20p-PABD colocalized with EGFP-DGKβ at the plasma membrane. However, DsRed-Spo20p-PABD was localized at the plasma membrane even in the absence of EGFP-DGKβ. We confirmed that DsRed-PDE4A1-PABD, which was reported to localize in the Golgi apparatus, was primarily distributed in the peri-nuclear region in the absence of EGFP-DGKβ(FIG. 28). Even when EGFP-DGKβ was expressed, DsRed-PDE4A1-PABD still existed in the Golgi apparatus, and its colocalization with EGFP-DGKβ was not observed.

### (3) Colocalization of α-Syn-N with Myristoylated DGKζ in the Plasma Membrane and Golgi Apparatus in COS-7 Cells

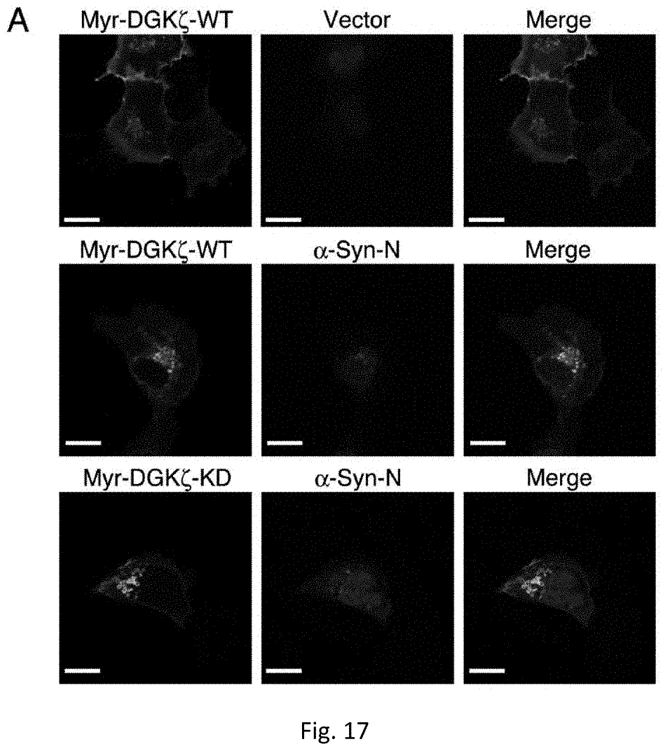

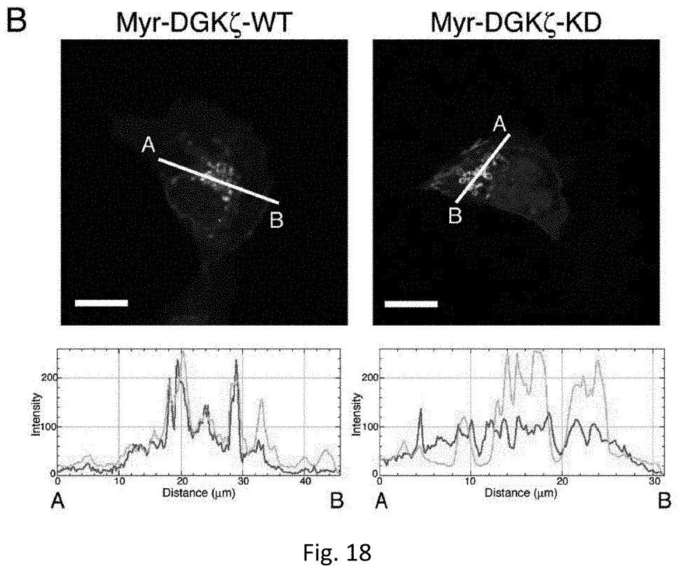

DGKζ was broadly localized in the cytoplasm and nucleus. To produce PA, DGKζ is needed to translocate to membranes where its substrate exists. It is known that myristoylation facilitates membrane localization. Thus, to induce the membrane localization of DGKζ, we generated AcGFP-DGKζ cDNA that possesses a consensus sequence of c-Src, serving as a target of N-myristoylation at its 5′ end (Myr-AcGFP-DGKζ). We verified that Myr-AcGFP-DGKζ was primarily localized in the perinuclear region and the plasma membrane (FIGS. 17, 18). The localization pattern of Myr-AcGFP-DGKζ overlapped with that of a trans Golgi network marker, TGN46, and a Golgi-localizing protein, DsRed-PDE4A1-PABD, indicating that Myr-AcGFP-DGKζ was distributed in the Golgi apparatus. DsRed-α-Syn-N markedly colocalized with Myr-AcGFP-DGKζ at the trans Golgi network and the plasma membrane (FIGS. 17, 18). However, its kinase-dead mutant (Myr-AcGFP-DGKζ-KD) did not show such overlap, demonstrating that the overlap depends on the activity of Myr-DGKζ to produce PA. Moreover, these results indicate that DsRed-α-Syn-N can track PA not only in the plasma membrane but also in the Golgi apparatus.

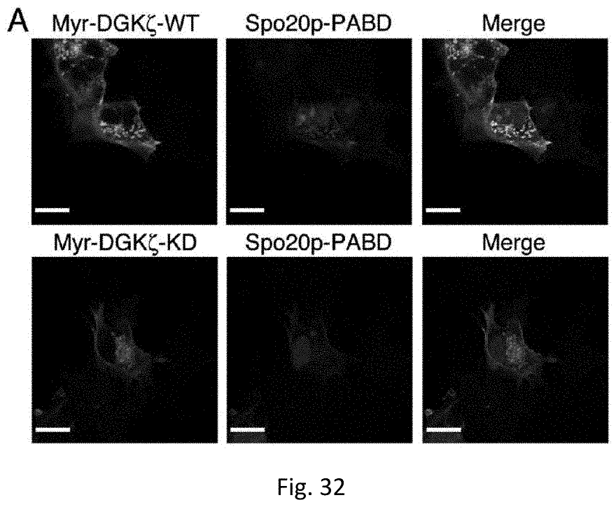

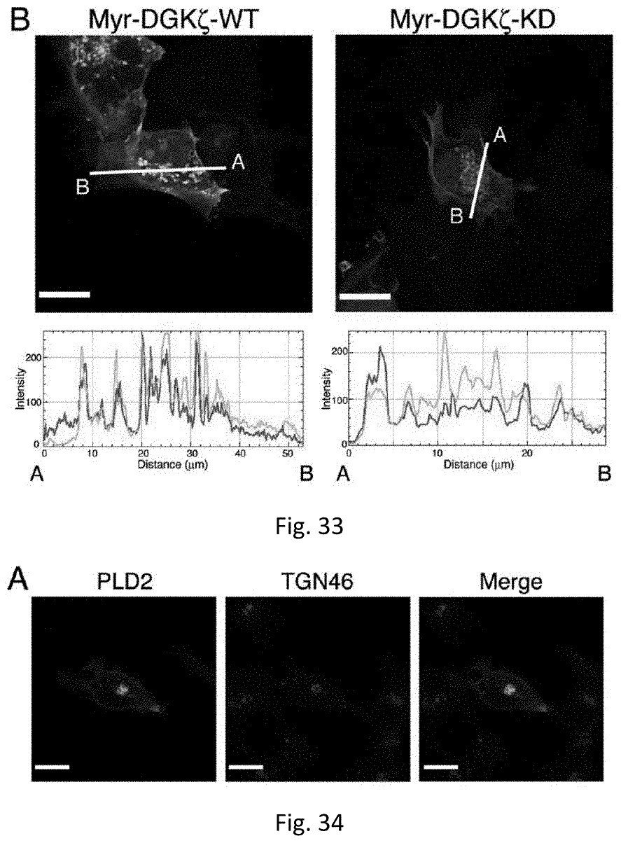

Spo20p-PABD also colocalized with Myr-AcGFP-DGKζ, but not with Myr-AcGFP-DGKζ-KD, at the Golgi apparatus (trans Golgi network) in COS-7 cells (FIGS. 32, 33), indicating that the PABD can also track PA in the Golgi apparatus. However, Spo20p-PABD was also colocalized with Myr-AcGFP-DGKζ-KD at the plasma membrane. Therefore, it is difficult to detect PA in the plasma membrane of COS-7 cells using Spo20p-PABD.

### (4) Colocalization of α-Syn-N with DGKγ in Phorbol Myristate Acetate-Stimulated COS-7 Cells

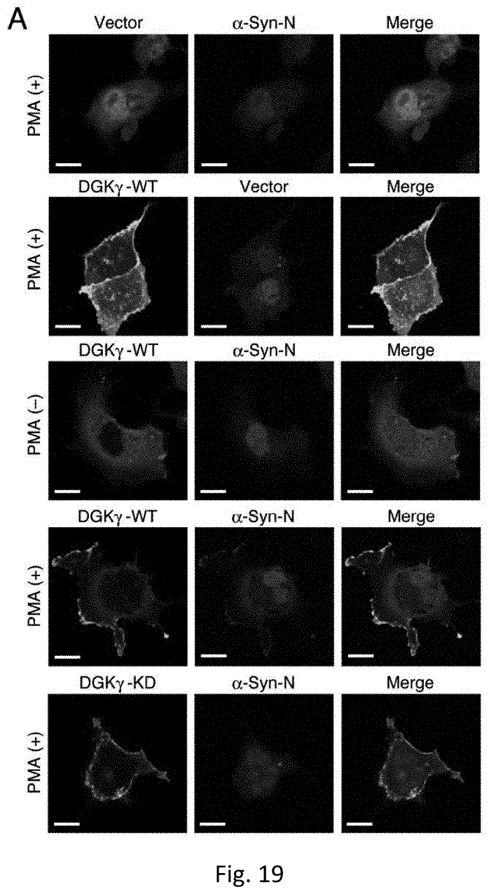

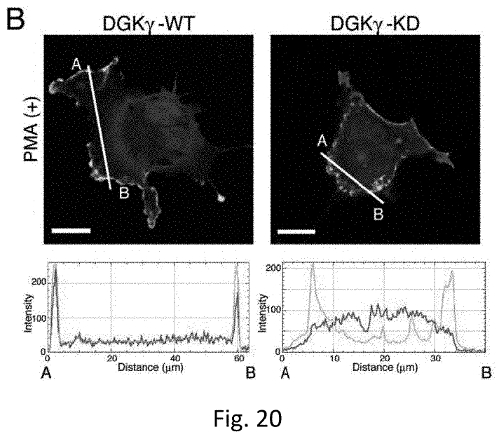

We next examined whether DsRed-α-Syn-N is able to detect PA that is produced for only a short term. DGKγ was reported to translocate from the cytoplasm to the plasma membrane, where its substrate exists, by phorbol 12-myristate 13-acetate (PMA) stimulation. Thus, COS-7 cells expressing EGFP-DGKγ were treated with PMA for 30 min. We confirmed that PMA stimulation led to the translocation of EGFP-DGKγ from the cytoplasm to the plasma membrane (FIG. 19). Notably, DsRed-α-Syn-N markedly colocalized with EGFP-DGKγ at the plasma membrane after PMA stimulation (FIGS. 19, 20). However, DsRed-α-Syn-N was not codistributed with DGKγ-KD, a kinase-dead inactive mutant, even after PMA stimulation, showing that DGK activity (PA production) is necessary for the colocalization. Therefore, these results strongly suggest that α-Syn-N is able to detect PA that is produced for a short term.

### (5) Colocalization of α-Syn-N with PLD2 in COS-7 Cells

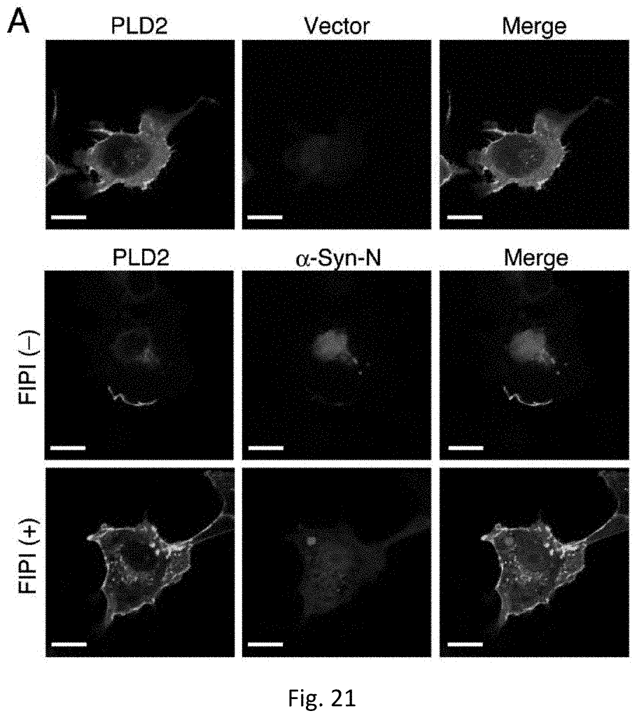

We next utilized PLD2 instead of DGK as a PA-generating enzyme. EGFP-PLD2 was distributed to the plasma membrane and Golgi apparatus in COS-7 cells. DsRed-α-Syn-N, but not with DsRed alone, clearly colocalized with EGFP-PLD2 (FIGS. 21, 34). Furthermore, a PLD inhibitor, 5-fluoro-2-indolyl deschlorohalopemide (FIPI), markedly attenuated their colocalization, indicating that the co-localization is PLD activity (PA generation)-dependent. These results indicate that α-Syn-N can recognize PA produced by all PA-producing enzymes investigated here at various membrane environments.

### (6) No Colocalization Between α-Syn-N and PIP5K1A in COS-7 Cells

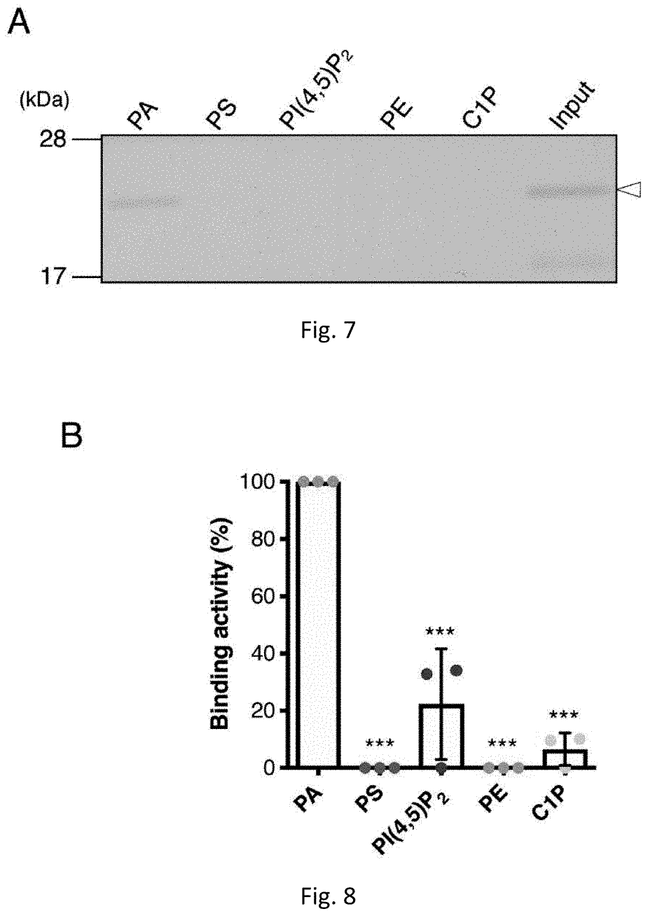

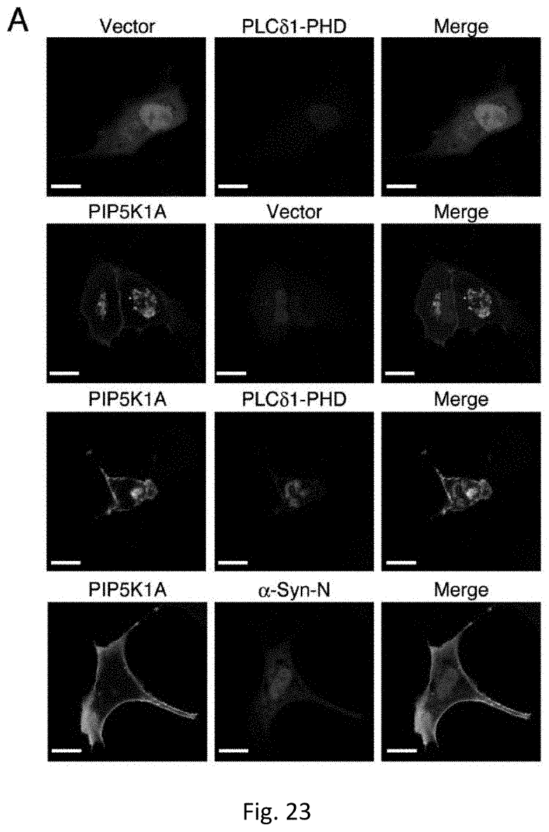

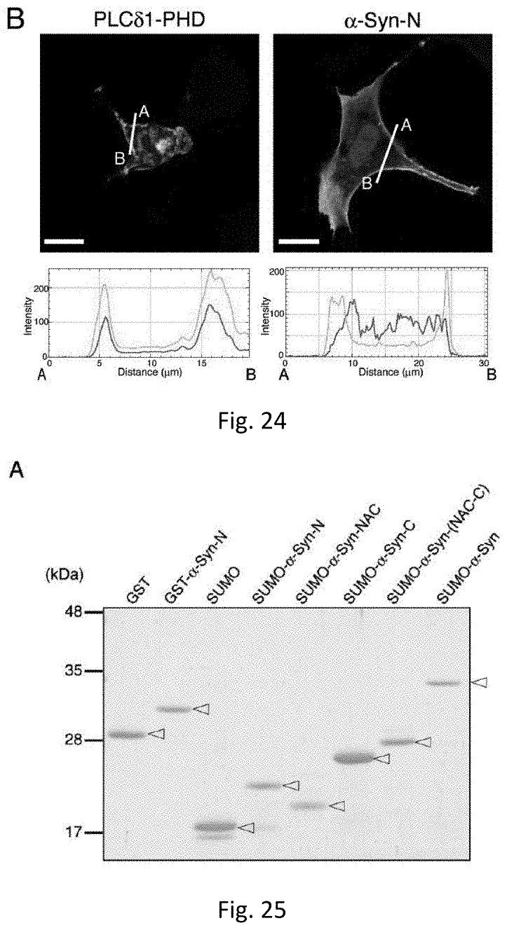

Because weak signals of α-Syn-N were detected at the PI(4,5)P₂-spot and in the PI(4,5)P₂-liposome (FIGS. 7-12), we next examined whether α-Syn-N colocalized with the PI(4,5)P₂-production enzyme, PIP5K1A, in COS-7 cells. DsRed-α-Syn-N did not codistribute with EGFP-PIP5K1A (FIGS. 23, 24). We confirmed that DsRed-phospholipase C (PLC) δ1-pleckstrin homology domain (PHD), which has been established as a PI(4,5)P₂ sensor, colocalized with PIP5K1A(FIGS. 23, 24). These results indicate that α-Syn-N does not recognize PI(4,5)P₂ and specifically recognizes PA in cells.

### (7) Discussion

There have been no reliable and widely applicable PA sensors to date. Although Spo20p-PABD and PDE4A1-PABD are often used as intracellular PA sensors, they show their own subcellular localization to the plasma membrane (Spo20p-PABD) and Golgi apparatus (PDE4A1-PABD) independent of cell stimulation-induced PA generation (in quiescent cells). In the present study, we revealed α-Syn-N as a novel, reliable and widely applicable PA sensor. Notably, DsRed monomer-α-Syn-N did not exhibit PA generation-independent, nonspecific subcellular localization in cells because its localization was essentially the same as the DsRed monomer, which is an essential property for an excellent lipid sensor. Moreover, the result further suggests that, in addition to PI(4,5)P₂ (FIG. 10), α-Syn-N does not recognize major phospholipids such as PC, PS, PE and PI, which showed no significant association in vitro (FIGS. 7-12), in cells. Moreover, α-Syn-N actually translocated to different membranes (the plasma membrane and Golgi apparatus) where active PA-generating enzymes, DGKβ, Myr-DGKζ, PMA-stimulated DGKγ and PLD2, were located (FIGS. 13-22). However, α-Syn-N failed to colocalize with them in the presence of their inhibitors and/or with their inactive mutants. These results indicate that α-Syn-N successfully tracked PA produced there. Moreover, α-Syn-N could detect PA generated by DGKγ that was translocated by PMA and existed for a short term (30 min) (FIGS. 19, 20), in addition to steady-state PA production by PA-generating enzymes for more than 24 h.

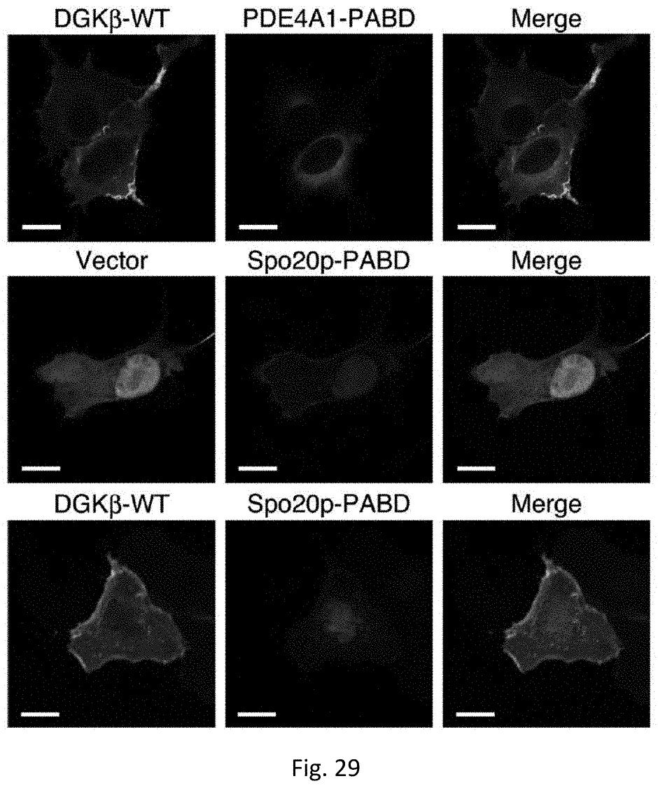

Spo20p-PABD and PDE4A1-PABD localized to the plasma membrane/nucleus and Golgi apparatus, respectively. In the present study, we reproduced the subcellular localization of these PABDs (FIGS. 29-34). The K_{d} value of α-Syn-N for PA (6.6 μM) was comparable with that of Spo20p-PABD (K_{d}=2.2 μM) and PDE4A1-PABD (K_{d}=6.8 μM). Therefore, α-Syn-N is highly sensitive to PA, and its sensitivity is almost equal to that of Spo20p-PABD and PDE4A1-PABD. However, from the perspective of negligible nonspecific (PA production enzymeindependent) subcellular localization, it is likely that α-Syn-N is superior to Spo20p-PABD and PDE4A1-PABD. For example, Spo20-PABD could not detect PA that was forced to increase by DGKβ at the plasma membrane because the sensor was strongly located at the plasma membrane even in the absence of DGKβ (FIG. 29). Moreover, PDE4A1-PABD was not able to detect PA that was forced to increase by Myr-DGKζ at the Golgi apparatus because the sensor was highly localized at the Golgi apparatus even in the absence of Myr-DGKζ (FIGS. 29-31). On the other hand, α-Syn-N does not have its own subcellular distribution ability to membranes, such as the plasma membrane and Golgi apparatus (FIGS. 13-16). Therefore, α-Syn-N would easily detect PA production at any cellular membranes. This point is highly advantageous.

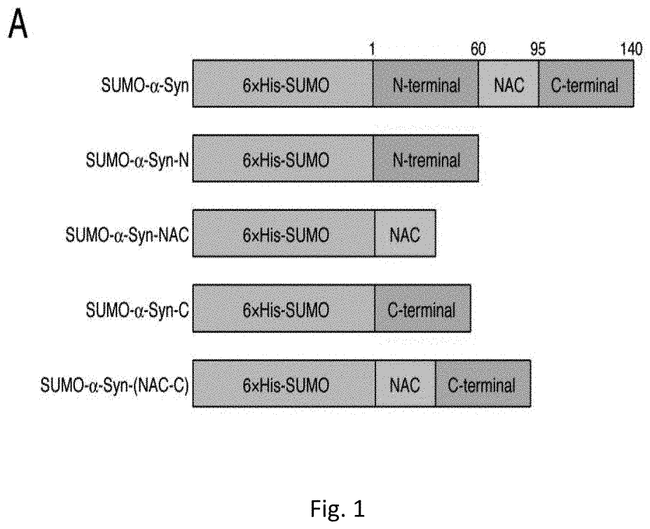

The experiments using the deletion mutants of α-Syn showed that the N-terminal region (α-Syn-N), but not the non-amyloid-β-component (α-Syn-NAC) or the C-terminal region (α-Syn-C), strongly bound to PA (FIGS. 1, 2). Consistent with a previous report using full-length α-Syn, α-Syn-N exhibited relatively broad selectivity for PA species, including 18:1/18:1-PA, 16:0/18:1- and 16:0/16:0-PA (FIGS. 3, 4). We recently demonstrated that DGKδ produced 14:0/16:0-, 14:0/16:1-, 16:0/16:0-, 16:0/16:1-, 16:0/18:0- and 16:0/18:1-PA, in glucose-stimulated C2C12 cells. Moreover, the production of 16:0/16:0- and 16:0/18:0-PA species was attenuated by DGKα-specific inhibitors. DGKζ exclusively generates very restricted PA species, 16:0/16:0-PA, during the initial/early stage of neuroblastoma cell differentiation. PLD hydrolyzes PC, which contains large amounts of 16:0/18:1- and 18:1/18:1-PA. Because LPAAT is involved in the de novo synthesis of major phospholipids, this enzyme generates broad PA molecular species. Therefore, α-Syn-N can be used as a PA sensor in a wide variety of cell lines treated with many kinds of cell stimulation because of its relatively broad selectivity for PA molecular species.

Inhibitor/activator screening of chemical compound libraries that targets enzymes in cells is generally more difficult than screening in vitro. α-Syn-N could be applied to screening of inhibitors/activators of PA-generating enzymes, including DGK, PLD and LPAAT, in cells. Several DGK isozymes can serve as potential drug targets for cancer, epilepsy, obsessive-compulsive disorder, bipolar disorder, autoimmunity, cancer immunity, cardiac hypertrophy, hypertension and type II diabetes. In addition, PLD is a drug target for cancer and neurodegenerative diseases, such as Parkinson's and Alzheimer's diseases. LPAAT was also reported to be a therapeutic target in gynecologic and lymphoid malignancies. As a PA sensor, α-Syn-N would be a useful tool for screening of inhibitors/activators of PA-generating enzymes to develop drugs for the variety of diseases mentioned above. Indeed, we successfully detected the effects of a DGKβ inhibitor, Compound A, and a PLD inhibitor, FIPI, in COS-7 cells (FIGS. 13-16, 21, 22).

In summary, we demonstrated that α-Syn-N is a promising cellular PA sensor that is reliable and applicable for a wide variety of physiological events. Thus, α-Syn-N was added to the list of cellular PA sensors. The utilization of multiple sensors for PA expands the application range and increases the reliability of the results of PA sensing. Moreover, α-Syn-N would be useful for the screening of inhibitors/activators of PA-generating enzymes, such as DGK, PLD and LPAAT, which are promising drug targets for a wide variety of diseases.

### (8) Figure Legends

FIG. 1. A schematic architecture of α-Syn and its deletion mutants. FIG. 2. The purified SUMO-tagged-α-Syn and its deletion mutant protein (2.5 μM) were incubated with 18:1/18:1-PA or 18:1/18:1-PS liposomes (PA or PA: 150 μM) and then separated by ultracentrifugation. SDS-PAGE (15%) was performed and separated proteins were stained with Coomassie Brilliant Blue. The positions of α-Syn and its mutants are indicated with arrow heads. The amounts of protein in the supernatant (S) and precipitant (P) were quantified by densitometry using ImageJ software. Binding activity was calculated as the percentage of the precipitate band intensity compared to the total band intensities. Values are presented as the mean±S.D. of four independent experiments. **P<0.01 versus the 18:1/18:1-PS liposome.

FIG. 3. The purified SUMO-tagged-α-Syn-N protein (2.5 μM) was incubated with control (PC and Chol alone), 16:0/16:0-PA, 16:0/18:1-PA, 18:1/18:1-PA, or 18:0/18:0-PA liposomes (PA: 150 μM) and then separated by ultracentrifugation. SDS-PAGE (15%) was performed and separated proteins were stained with Coomassie Brilliant Blue. The position of α-Syn-N is indicated with an arrow head.

FIG. 4. The amounts of protein in the supernatant (S) and precipitant (P) were quantified by densitometry using ImageJ software. Binding activity was calculated as the percentage of the precipitate band intensity compared to the total band intensities. Values are presented as the mean±S.D. of three independent experiments. #P<0.05, ##P<0.01, ###P<0.005 versus the 18:1/18:1-PA-liposome, *P<0.05, ***P<0.005 versus the control liposome.

FIG. 5. The purified SUMO-tagged-α-Syn protein (0.1 μM) was incubated with 18:1/18:1-PA liposomes of the indicated concentrations (0-20 μM) and then separated by ultracentrifugation. SDS-PAGE (15%) was performed and separated proteins were detected with silver staining. The position of α-Syn-N is indicated with an arrow head.

FIG. 6. The amounts of protein in the precipitant were quantified by densitometry using ImageJ software. Binding activity was calculated as the percentage of the precipitate band intensity compared to the total band intensities (Input). Values are presented as the mean±S.D. of four independent experiments. The K_{d} was determined using GraphPad Prism 8 (Dissociation-One phase exponential decay).

FIG. 7. The purified SUMO-tagged-α-Syn protein (0.1 μM) was incubated with 18:1/18:1-PA, 18:1/18:1-PS, 18:1/18:1-PI(4,5)P₂, 18:1/18:1-PE and d18:1/18:1-C1P liposomes (20 μM) and then separated by ultracentrifugation. SDS-PAGE (15%) analysis was performed and separated proteins were detected with silver staining. The position of α-Syn-N is indicated with an arrow head.

FIG. 8. The amounts of protein in the precipitant were quantified by densitometry using ImageJ software. The PA binding activity of α-Syn-N was set to 100%. Values are presented as the mean±S.D. of three independent experiments. ***P<0.005 versus 18:1/18:1-PA liposome.

FIG. 9. Equimolar amounts (100 pmol) of various lipids were spotted onto nitrocellulose membranes as indicated. Acyl chains of these glycerolipids and sphingolipids are C16:0 (Echelon Biosciences).

FIG. 11. Equimolar amounts (300 pmol) of 18:1/18:1-PA, 18:1-LPA, and d18:1/18:1-C1P were spotted onto nitrocellulose membranes as indicated. The membranes were incubated with purified GST-tagged-α-Syn-N(20 nM). Lipid-bound proteins were detected by an anti-GST antibody.

FIGS. 10 and 12. The blots were scanned and quantified using ImageJ software. The PA binding activity (spot intensity) of α-Syn-N was set to 100%. Values are presented as the mean±S.D. of three independent experiments. ***P<0.005 versus PA.

FIGS. 13 to 16. Either EGFP alone, EGFP-DGKβ-WT or EGFP-DGKβ-KD was coexpressed with either the DsRed monomer alone or DsRed monomer-α-Syn-N in COS-7 cells as indicated. After 24 h of transfection, the cells were fixed and imaged. Representative data from three independent experiments are shown. Scale bars, 20 μm.

FIG. 14. The localization of EGFP-DGKβ-WT, EGFP-DGKβ-KD and DsRed monomer-α-Syn-N was quantified using ImageJ software. FIG. 15. EGFP-DGKβ-WT was coexpressed with DsRed monomer-α-Syn-N in COS-7 cells. After 24 h of transfection, the cells were incubated with 1 μM Compound A in growth medium for 1 h, fixed and imaged. Representative data from three independent experiments are shown. Scale bars, 20 μm.

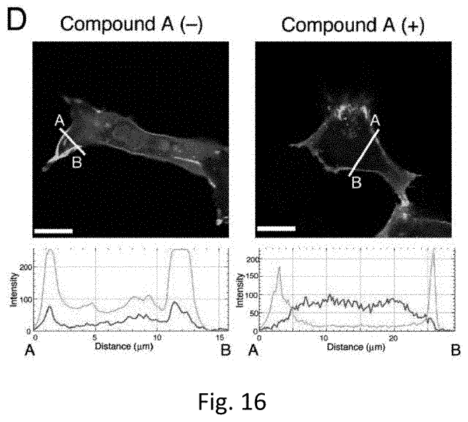

The lower graph in FIG. 16 is the localization of EGFP-DGKβ-WT and DsRed monomer-α-Syn-N was quantified using ImageJ software.

FIG. 17. Either Myr-AcGFP-DGKζ-WT or Myr-AcGFP-DGKζ-KD was coexpressed with either the DsRed monomer alone or DsRed monomer-α-Syn-N in COS-7 cells as indicated. After 24 h of transfection, the cells were fixed and imaged. Representative data from three independent experiments are shown. Scale bars, 20 μm.

FIG. 18. The localization of Myr-AcGFP-DGKζ-WT, Myr-AcGFP-DGKζ-KD and DsRed monomer α-Syn-N was quantified using ImageJ software.

FIGS. 19 and 20. Either EGFP alone, EGFP-DGKγ-WT or EGFP-DGKγ-KD was coexpressed with either the DsRed monomer alone or DsRed monomer-α-Syn-N in COS-7 cells as indicated. After 24 h of transfection, the cells were incubated with 1 μM PMA or DMSO alone in growth medium for 30 min, fixed and imaged. Representative data from three independent experiments are shown. Scale bars, 20 μm. FIG. 20. The localization of EGFP-DGKγ-WT, EGFP-DGKγ-KD and DsRed monomer α-Syn-N was quantified using ImageJ software.

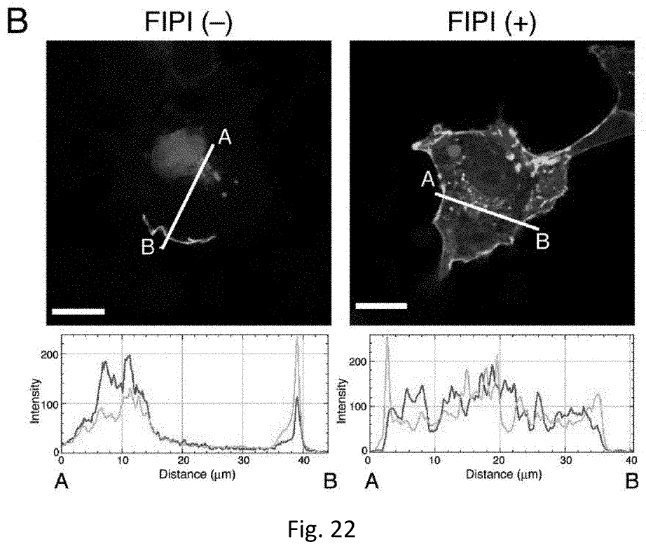

FIGS. 21 and 22. EGFP-PLD2 was coexpressed with either the DsRed monomer alone or DsRed monomer α-Syn-N in COS-7 cells as indicated. After 24 h of transfection, the cells were incubated with 750 nM FIPI in growth medium for 4 h and then fixed and imaged. Representative data from three independent experiments are shown. Scale bars, 20 μm.

FIG. 22. The localization of EGFP-PLD2-WT and DsRed monomer α-Syn-N was quantified using ImageJ software.

FIG. 23. Either EGFP alone or EGFP-PIP5K1A was coexpressed with either the DsRed monomer alone, DsRed monomer α-Syn-N or DsRed monomer PLCδ1-PHD in COS-7 cells as indicated. After 24 h of transfection, the cells were fixed and imaged. Representative data from three independent experiments are shown. Scale bars, 20 μm.

FIG. 24. The localization of EGFP-PIP5K1A, DsRed monomer-α-Syn-N and DsRed monomer-PLCδ1-PHD was quantified using ImageJ software.

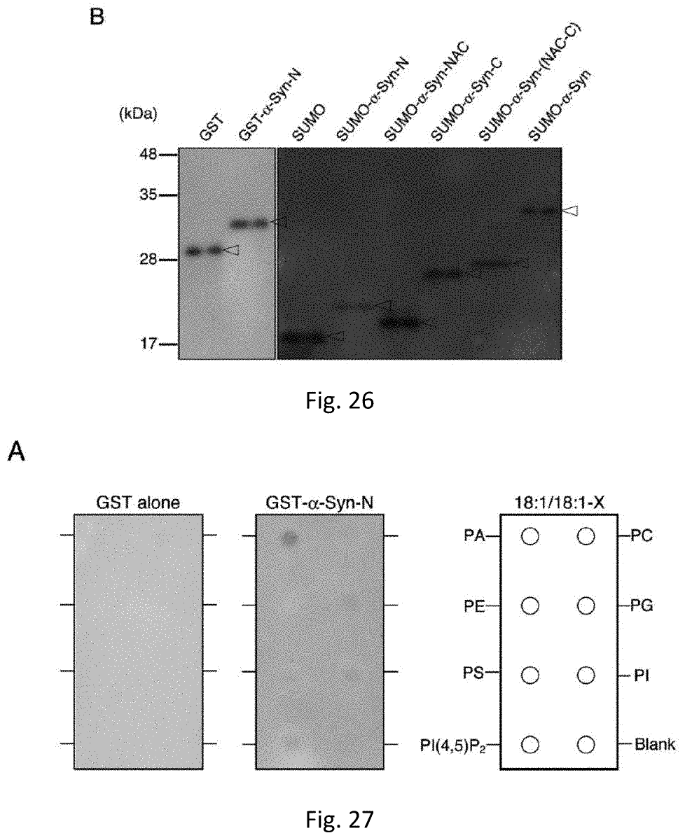

FIGS. 25 and 26. SDS-PAGE (15%) analysis of GST or SUMO-tagged α-Syn and its mutants expressed in E. coli and purified by affinity chromatography. Separated proteins were stained with Coomassie Brilliant Blue. The positions of α-Syn and its mutants, which were determined by immunoblot analysis using anti-GST and anti-6×His antibodies (FIG. 26), are indicated with arrow heads. Expected molecular masses: GST alone, 28.4 kDa; GST-α-Syn-N, 31.7 kDa; SUMO alone, 12.2 kDa, SUMO-α-Syn-N, 18.4 kDa; SUMO-α-Syn-NAC, 15.5 kDa; SUMO-α-Syn-C, 17.3 kDa; SUMO-α-Syn-(NAC-C), 20.5 kDa; SUMO-α-Syn, 26.7 kDa.

FIG. 27. Equimolar amounts (500 pmol) of 18:1/18:1-PA, 18:1/18:1-PC, 18:1/18:1-PE, 18:1/18:1-PG), 18:1/18:1-PS, 18:1/18:1-PI and 18:1/18:1-PI(4,5)P₂ were spotted onto nitrocellulose membranes as indicated and incubated with purified GST-tagged-α-Syn-N(20 nM). Lipid-bound proteins were detected by an anti-GST antibody.

FIG. 28. The blots were scanned and quantified using ImageJ software. The PA binding activity (spot intensity) of α-Syn-N was set to 100%. Values are presented as the mean±S.D. of three independent experiments. ***P<0.005 versus 18:1/18:1-PA.

FIG. 29. Either EGFP alone or EGFP-DGKβ was coexpressed with the DsRed monomer alone, DsRed monomer-Spo20p-PABD or DsRed monomer-PDE4A1-PABD in COS-7 cells as indicated. After 24 h of transfection, the cells were fixed and imaged. Representative data from three independent experiments are shown. Scale bars, 20 μm.

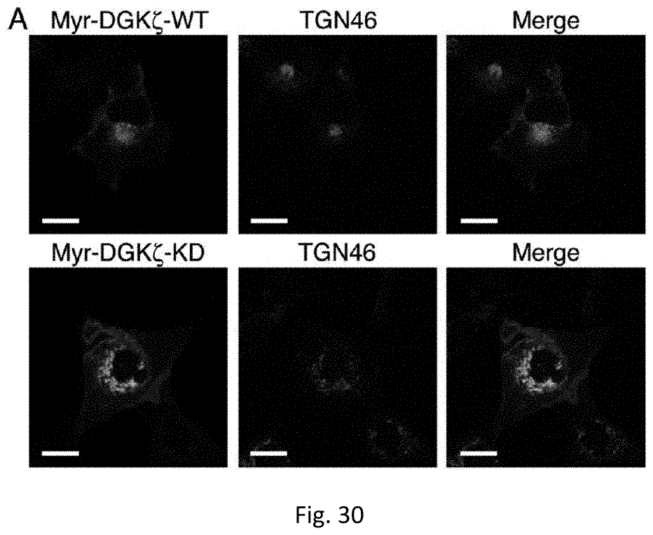

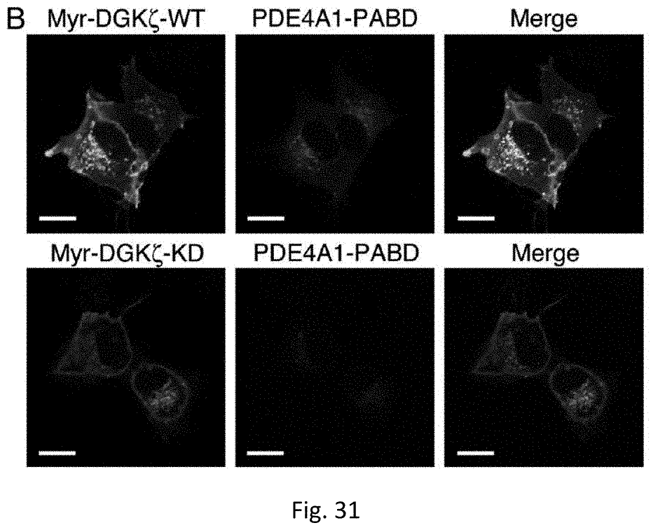

FIGS. 30 and 31. Myr-AcGFP-DGKζ and/or DsRed monomer-PDE4A1-PABD were expressed in COS-7 cells as indicated. After 24 h of transfection, the cells expressing Myr-AcGFP-DGK alone were immunostained with an anti-TGN46 antibody and an Alexa Fluor 568-conjugated secondary antibody. Representative images of three independent experiments are shown. Scale bars, 20 μm.

FIGS. 32 and 33. Either Myr-AcGFP-DGKζ alone or Myr-AcGFP-DGKζ-KD was coexpressed with the DsRed monomer alone or DsRed monomer-Spo20p-PABD in COS-7 cells as indicated. After 24 h of transfection, the cells were fixed and imaged. Representative data from three independent experiments are shown. Scale bars, 20 μm.

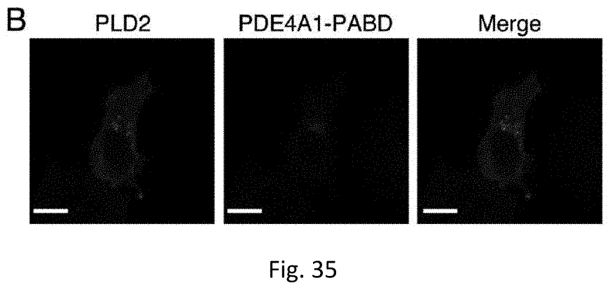

FIGS. 34 and 35. EGFP-PLD2 and/or DsRed monomer-PDE4A1-PABD were expressed in COS-7 cells as indicated. After 24 h of transfection, the cells expressing Myr-AcGFP-DGKζ alone were immunostained with an anti-TGN46 antibody and an Alexa Fluor 594-conjugated secondary antibody. Representative images of three independent experiments are shown. Scale bars, 20 μm.

### (9) Experimental Procedures

### (a) Expression and Purification of SUMO, and GST-Fused α-Syn and its Deletion Mutants.

To prepare 6×His-SUMO-fused constructs, the cDNAs of α-Syn (Met1-Ala140), α-Syn-N(Met1-Lys60), α-Syn-NAC (Glu61-Val95), α-Syn-C (Lys96-Ala140), and α-Syn-(NAC-C) (Glu61-Ala140), were amplified by PCR from the pET-14b-mouse α-Syn, and inserted into the pSUMO vector. The resulting plasmids were used to transform Escherichia coli strain Rosetta-gami 2 (DE3). Cells were cultured in LB media at 37° C. until the OD₆₀₀ reached 0.6-0.8. Expression of the recombinant protein was then induced by adding 0.1 mM isopropyl β-D-thiogalactopyranoside, and the bacterial culture was continued at 37° C. for 3 h. Bacteria cells harvested by centrifugation were suspended in lysis buffer (50 mM sodium phosphate, pH 8.0, containing 300 mM NaCl, 10 mM imidazole, 20 μg/mL aprotinin, 20 μg/mL leupeptin, and 20 μg/mL pepstatin) and lysed by sonication on ice. After cell lysis and centrifugation, Ni-affinity chromatography using Ni-NTA agarose was performed to purify 6×His-SUMO-fused proteins. The column was washed with 50 mM sodium phosphate, pH 8.0, 300 mM NaCl, and 10 and 50 mM imidazole, and the bound proteins were eluted with 300 mM imidazole. For the liposome cosedimentation assay, the purified proteins were dialyzed in HEPES buffer (25 mM HEPES, pH 7.4, 100 mM NaCl). The protein concentration was measured with a bicinchoninic acid protein assay kit (Thermo Fisher Scientific).

For GST-tagged α-Syn-N, the DNA sequence of α-Syn-N (Met1-Lys60) was amplified by PCR from the pET-14b-mouse α-Syn and inserted into the BamHI and SalI restriction sites of the pGEX-6P-1 vector. The resulting plasmids were used to transform E. coli strain Rosetta 2(DE3). Cells were cultured in LB media at 37° C. until the OD₆₀₀ reached 0.6-0.8. Expression of the recombinant protein was then induced by adding 0.1 mM isopropyl β-D-thiogalactopyranoside, and the bacterial culture was continued at 37° C. for 3 h. Bacteria harvested by centrifugation were suspended in lysis buffer (1.47 mM KH₂PO₄, 8.09 mM Na₂HPO₄, pH 7.2, 2.67 mM KCl, 137 mM NaCl, 20 μg/mL aprotinin, 20 μg/mL leupeptin, and 20 μg/mL pepstatin) and lysed by sonication on ice. After cell lysis and centrifugation, affinity chromatography using glutathione-Sepharose 4B was carried out to purify GST-fused α-Syn-N. The column was washed with lysis buffer and the bound protein was eluted with 50 mM Tris-HCl, 150 mM NaCl, and 10 mM reduced glutathione. For the lipid overlay assay, the purified proteins were dialyzed in HEPES buffer. The protein concentration was measured with a bicinchoninic acid protein assay kit (Thermo Fisher Scientific).

### (b) Liposome Cosedimentation Assay

The following lipid mixtures were used: the control liposome: Chol (50 mol %) and 16:0/18:1-PC (50 mol %); and the PA liposome: Chol (50 mol %), 16:0/18:1-PC (35 mol %) and each PA species (15 mol %). The combined dried lipid mixture was resuspended in HEPES buffer. Then, the lipid mixture was vortexed for 1 min. Liposome formation was induced by sonication at 75° C. The purified SUMO-tagged α-Syn and its deletion mutants (2.5 μM) were dissolved in HEPES buffer and incubated with the control or PA-containing liposomes at room temperature for 1 h. Then, the samples were centrifuged at 200,000 g at 4° C. for 1 h. The precipitant was dissolved in HEPES buffer. The supernatant and pellet were analyzed by SDS-PAGE followed by Coomassie Brilliant Blue and silver staining. Quantitative densitometry was performed using ImageJ software. Since the lipid forms a bilayer, half of the actual concentration was considered.

### (c) Lipid Overlay Assay

Three hundred picomoles of the indicated lipids were spotted onto a nitrocellulose membrane. Membrane Lipid Strip was also used. The membranes were subjected to blocking with 1% skim milk in Tris-buffered saline, pH 7.4, for 1 h at room temperature. After blocking, 10 ml of 3% bovine serum albumin in Tris-buffered saline, pH 7.4, containing purified GST alone or GST-α-Syn-N (final concentration, 20 nM) was added to the membranes. The membranes were then incubated for 1 h at room temperature. Next, the membranes were incubated with an anti-GST antibody, followed by incubation with a peroxidase-conjugated goat anti-rabbit IgG antibody. Finally, lipid-bound proteins were visualized using an ECL Western blotting detection system. Quantitative densitometry was performed using ImageJ software.

### (d) Cell Culture

COS-7 cells were maintained in Dulbecco's modified Eagle's medium supplemented with 10% fetal bovine serum, 100 units/ml penicillin, and 100 μg/ml streptomycin at 37° C. in an atmosphere containing 5% CO₂. Cells were transiently transfected using PolyFect reagent as described by the manufacturer.

### (e) Confocal Laser Scanning Microscopy

COS-7 cells were grown and transfected as described in the above section. After 24 h of transfection, pEGFP-DGKβ-transfected COS-7 cells were incubated with Compound A in DMEM (final concentration, 1 μM) for 1 h to inhibit DGKβ activity. pEGFP-PLD2-transfected cells were incubated with FIPI in growth medium (final concentration, 750 nM) for 4 h to inhibit PLD2 activity. The cells were then fixed in 4% paraformaldehyde. The coverslips were mounted using Vectashield. Fluorescence images were acquired using an Olympus FV1000-D (IX81) confocal laser scanning microscope. GFP fluorescence was excited at 488 nm, and DsRed fluorescence was excited at 543 nm. Images were acquired using FV-10 ASW software.

### (10) Statistical Analysis

Data are represented as the means±S.D. and were analyzed by the Student's t-test for the comparison of two groups or one-way ANOVA followed by Tukey's post hoc test for multiple comparisons using GraphPad Prism 8.

### INDUSTRIAL APPLICABILITY

The present invention can be used as an intracellular phosphatidic acid sensor.

What is claimed is:

1. A phosphatidic acid sensor comprising an N-terminal region of α-synuclein.

2. A phosphatidic acid sensor comprising a peptide having an amino acid sequence encoded by any one of following base sequences (1) to (3):
(1) a base sequence of SEQ ID No. 1;
(2) a base sequence having 90% or more homology to the base sequence of SEQ ID No. 1; and
(3) a base sequence in which 18 or less bases are deleted, substituted, and/or added to the base sequence of SEQ ID No. 1.

3. A phosphatidic acid sensor comprising a peptide having any one of following amino acid sequences (1) to (3):
(1) an amino acid sequence of SEQ ID No. 2;
(2) an amino acid sequence having 90% or more homology to the amino acid sequence of SEQ ID No. 2; and
(3) an amino acid sequence in which 6 or less amino acids are deleted, substituted, and/or added to the amino acid sequence of SEQ ID No. 2.

4. The phosphatidic acid sensor of claim 2, wherein the peptide having an amino acid sequence is encoded by a base sequence of SEQ ID No. 1.

5. The phosphatidic acid sensor of claim 3, wherein the peptide has an amino acid sequence of SEQ ID No. 2.

6. The phosphatidic acid sensor of claim 2, wherein the peptide having an amino acid sequence is encoded by a base sequence having 90% or more homology to the base sequence of SEQ ID No. 1.

7. The phosphatidic acid sensor of claim 3, wherein the peptide has an amino acid sequence having 90% or more homology to the amino acid sequence of SEQ ID No. 2.

8. The phosphatidic acid sensor of claim 2, wherein the peptide having an amino acid sequence is encoded by a base sequence in which 18 or less bases are deleted, substituted, and/or added to the base sequence of SEQ ID No. 1.

9. The phosphatidic acid sensor of claim 3, wherein the peptide has an amino acid sequence in which 6 or less amino acids are deleted, substituted, and/or added to the amino acid sequence of SEQ ID No. 2.
